(19) United States
(12) Patent Application Publication
(10) Pub. No.: US 2020/0291484 A1
(43) Pub. Date: Sep. 17, 2020

(54) METHODS OF DETECTING MINIMAL RESIDUAL DISEASE

(71) Applicant: Stitch Bio, LLC, Waltham, MA (US)

(72) Inventor: Anthony P. Shuber, Northbridge, MA (US)

(21) Appl. No.: 16/816,627

(22) Filed: Mar. 12, 2020

Related U.S. Application Data
(60) Provisional application No. 62/818,981, filed on Mar. 15, 2019.

(51) Int. Cl. C12Q 1/6886; C12Q 1/6844; C12N 9/22

(52) U.S. Cl. CPC C12Q 1/6886; C12N 9/22; C12Q 1/6844

(57) ABSTRACT

The invention provides methods of detecting residual disease, such as cancer, in a subject. The methods entail amplifying from a sample obtained from the subject one or more nucleic acids having a passenger mutation that contains or results from fusion, such as a translocation, inversion, or deletion.

### FIELD OF THE INVENTION

The invention relates to molecular diagnostics.

### BACKGROUND

An inherent problem with most cancer therapies is that they cannot achieve complete elimination of cancer cells from the body. Although treatment may resolve all clinical signs of cancer in a given patient, the survival of a very small number of residual cancer cells may eventually cause the disease to return. The rates of cancer recurrence vary widely depending on factors such as cancer type, stage of initial diagnosis, and patient related variables. For several types of cancer, such as glioblastoma, ovarian cancer, and certain types of lymphoma, recurrence rates exceed 50%.

Effective monitoring of residual disease in patients with cancer requires methods that can detect tumor cells that represent a tiny fraction of the affected tissue. DNA-based tests hold potential for cancer screening because a hallmark of cancer cells is their genetic instability. Mutations that confer a selective growth advantage on cells, called “driver” mutations, lead to cancer, and most tumors have multiple driver mutations. Therefore, one approach is to screening for surviving cancer cells is to analyze an individual's DNA using panels of known driver mutations. However, results from such screens are seldom conclusive because only a small subset of driver mutations is present in a typical tumor, and residual cancer cells may go undetected in a patient whose test results are negative for a given suite of driver mutations. Consequently, current methods of DNA-based screening for residual cancer cells are inadequate, and even patients who have undergone successful treatment live with the specter that their cancer could return.

### SUMMARY

The invention provides methods for detecting residual cancer cells in a patient by screening for “passenger” mutations, i.e., mutations that have no effect on the neoplastic process, that result from genetic fusion events. Passenger mutations are far more abundant in cancer cells than are driver mutations. Therefore, the invention recognizes that screening cancer patients for passenger mutations provides a broader sampling of tumor-associated genetic changes and permits detection of residual cancer cells that would be missed by methods that rely on driver mutations or other cancer biomarkers. In addition, by looking for evidence of genetic instability rather than events that trigger neoplasticity, methods of the invention allow residual cancer cells to be detected at an earlier stage, enabling more accurate assessment of the patient's risk of recurrence and more effective therapeutic intervention.

Methods of the invention entail detection of passenger mutations that contain or result from fusions, such as translocations, inversion, and deletions. For example, the methods include PCR amplification of fusion sites, which are only present in cells that have undergone genetic recombination. By focusing on fusion passenger mutations, methods of the invention provide binary results that indicate whether residual cancer cells are present in the patient's body. Additionally, the methods are quick and do not require expensive, specialized equipment. Moreover, the methods can use tissue samples, such as blood samples, that can be readily obtained from patients using non-invasive procedures. Consequently, the methods of the invention allow simple, rapid, and convenient screening for residual disease in cancer patients.

In an aspect, the invention provide methods of detecting residual cancer in a subject. The methods include obtaining a sample from a subject that has been treated for cancer and amplifying from the sample one or more nucleic acids that include a passenger mutation that contains or results from a fusion. For example, the passenger mutation may be or contain a deletion, inversion, or translocation.

The nucleic acid may be DNA or RNA. The nucleic acid may by a subpopulation of DNA or RNA from the sample. For example, the DNA may be cell-free DNA (cfDNA), circulating tumor DNA (ctDNA), or circulating cell-free mitochondrial DNA (ccf mtDNA). The RNA may mRNA, tRNA, rRNA, or snRNA.

The passenger mutation may be amplified using a pair of primers that bind to regions in the nucleic acid that flank the fusion site. For example, one primer may be complementary to a sequence in the nucleic acid on one side of the fusion, and the other primer may be complementary to a sequence in the nucleic acid on the other side of the fusion. The passenger mutation may be amplified by the polymerase chain reaction (PCR).

Any sample from the subject may be used for analysis. For example, the sample may be or include one or more of bile, blood, bone marrow, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, and vomit. The sample may be obtained by a liquid biopsy.

The method may include multiplexing, i.e., amplifying multiple nucleic acids in a single reaction or assay. For example, the method may include amplifying 2, 3, 4, 5, 6, 7, 8, 9, 10, 11, 12, 13, 14, 15, 16, 17, 18, 19, 20, about 25, about 30, about 40, about 50, or more nucleic acids. Each nucleic acid may contain a passenger mutation containing or resulting from a fusion. Each nucleic acid may contain a different passenger mutation. Alternatively or additionally, two or more of the nucleic acids may contain the same passenger mutation, i.e., two or more of the nucleic acids may overlap.

The subject may have any type of cancer. The cancer may be associated with one or more passenger mutations that contains or results from a fusion. For example, the cancer may be breast cancer, colon cancer, gastric cancer, glioblastoma, leukemia, liposarcoma, liver cancer, lung cancer, lymphoma, medullablastoma, melanoma, oligoastrocytoma, oligodendroglioma, ovarian cancer, pancreatic cancer, prostate cancer, sarcoma, or thyroid cancer.

The method may include detecting a nucleic acid. The method may include detecting a nucleic acid after amplification. The nucleic acid may be detected by electrophoresis, chromatography, or fluorescence.

The method may include enriching the sample for a nucleic acid. The nucleic acid may be enriched using a Cas endonuclease and a guide RNA. The enrichment may be performed prior to the amplification, or it may be performed after the amplification.

The method may include providing a report containing information about the residual cancer in the subject, prior treatment of the subject, or a subsequent course of treatment for the subject. For example, the report may contain information on one or more of the following: identify of fusion passenger mutations in the sample; number of fusion passenger mutations in the sample; estimate of the subject's tumor mutational burden; efficacy of the subject's prior treatment; development of resistance to a therapy or therapeutic agent in the subject's prior treatment; prognosis for course of disease in the subject; probability that the disease or a particular clinical manifestation of the disease will return for the subject; likelihood that subject will develop resistance to a subsequent therapy or therapeutic agent; and a suggested course of the therapy for the subject.

The one or more nucleic acids may have been previously detected in the subject. The one or more nucleic acids may not have been previously detected in the subject. The one or more nucleic acids may be detectable in a sample obtained from the subject prior to treatment of the subject for cancer, or they may not be detectable in a sample obtained from the subject prior to treatment of the subject for cancer. The one or more nucleic acids may be detectable in a sample obtained from the subject following treatment of the subject for cancer, or they may not be detectable in a sample obtained from the subject following treatment of the subject for cancer.

The passenger mutation that contains or results from a fusion may have been previously detected in one or more samples obtained from another subject or group of subjects. Alternatively, the passenger mutation may not have been previously detected in a sample obtained from another subject or group of subjects. The passenger mutation that contains or results from a fusion may have been previously detected in one or more samples obtained from the same subject. Alternatively, the passenger mutation may not have been previously detected a sample from the same subject.

The passenger mutation that contains or results from a fusion may be associated with cancer in the same subject. The passenger mutation that contains or results from a fusion may be associated with cancer in another subject or group of subjects.

### DETAILED DESCRIPTION

A challenge of treating cancer is that survival of a single tumor cell is sufficient to cause the disease to return. Cancer therapies are designed to kill or eliminate 100% of cancer cells, but even treatment methods that result in resolution of all clinical signs of the disease may still leave a handful of residual cancer cells in the patient's body. Because their proliferation is unregulated, the remaining cancer cells may expand in number and invade other tissues, resulting a recurrence of the disease. However, when cancer cells are few in number and have not metastasized, their presence is difficult to detect. Thus, for a cancer patient who has already achieved a defined benchmark from a course of therapy, it is often difficult to determine whether or when additional treatment is necessary.

The invention addresses the foregoing problem by providing methods for detecting residual cancer cells in a patient. The methods entail screening for specific markers of genetic instability, a signature trait of cancer cells. Because the markers are present only in genetically unstable cells, the methods allows detection of cancer cells even when they are present in the body in very low abundance. The methods allow a bodily fluid sample to be assayed for fusion passenger mutations in an inexpensive, quick, and reliable manner and thus are conducive to high throughput screening. In addition, the methods provide profiles of fusion passenger mutations specific to an individual. Therefore, they permit monitoring of cancer recurrence in an individual by detecting changes in an individual's profile of fusion passenger mutations over time.

Passenger Mutations that Contain or are Derived from DNA Fusions

Somatic mutations that arise due to the genetic instability of cancer cells fall into two functional categories. The first functional category includes mutations that confer a selective growth advantage drive tumorigenesis and thus are called “driver” mutations. Even though a driver mutation promotes uncontrolled growth of a cancer cell, a single driver mutation is usually not sufficient to convert a normal cell into a tumor cell. On the contrary, most cancer cells harbor from two to eight driver mutations. Vogelstein, et al., Cancer Genome Landscapes, Science. 2013 Mar. 29; 339(6127):1546-58, doi: 10.1126/science.1235122, the contents of which are incorporated herein by reference. The second category of functional mutations includes those that do not confer a selective growth advantage. Such mutations are called “passenger” mutations because they are “just along for the ride.” It is estimated that 97% of mutations in cancer are passengers. Lawrence M S, et al., Discovery and saturation analysis of cancer genes across tumor types, Nature, 2014 Jan. 23; 505(7484):495-501. doi: 10.1038/nature12912, the contents of which are incorporated herein by reference. Driver mutations and passenger mutations are known in the art and described in more detail in, for example, Vogelstein, et al., Cancer Genome Landscapes, Science. 2013 Mar. 29; 339(6127):1546-58, doi: 10.1126/science.1235122; Lawrence M S, et al., Discovery and saturation analysis of cancer genes across tumour types, Nature, 2014 Jan. 23; 505(7484):495-501. doi: 10.1038/nature12912; McFarland C D, et al., Cancer Res. 2017 Sep. 15; 77(18):4763-4772, doi: 10.1158/0008-5472.CAN-15-3283-T; and Pon, J R, and Marra, M A, Driver and passenger mutations in cancer, Annu Rev Pathol. 2015; 10:25-50. doi: 10.1146/annurev-pathol-012414-040312, the contents of each of which are incorporated herein by reference.

Somatic mutations in cancer cells also fall into two broad structural categories. In the first structural category are subtle somatic mutations that include small structural changes to DNA, such as single base substitutions and insertions or deletions of one or a few bases. Making up the second structural category are larger changes in chromosome structure, such as gene amplifications, deletions, inversions, and translocations. Such chromosomal rearrangements include fusions of DNA fragments that are not contiguous in wild-type or unaltered chromosomal DNA. Fusions are useful as targets for analysis by polymerase chain reaction (PCR) because primers that bind to targets on opposite sides of the fusion site typically only produce a product if the rearrangement has occurred.

Methods of the invention include analysis of nucleic acids that contain passenger mutations that contain or result from DNA fusions, i.e., fusion passenger mutations. The passenger mutation may contain or result from any type of fusion. For example and without limitation, the passenger mutation may contain or result from an amplification, deletion, duplication, insertion, inversion, or translocation.

The passenger mutation or collection of passenger mutations may be characteristic of a particular type of cancer. For example, the passenger mutation or collection of passenger mutations may have previously been detected in one or more reference subjects that have been diagnosed with a particular type of cancer.

The passenger mutation or collection of passenger mutations may be distinctive for an individual. For example, the passenger mutation or collection of passenger mutations may not have previously been detected in a reference subject. Thus, the passenger mutation or collection of passenger mutations may form a “signature’ for the test subject. The passenger mutation or collection of passenger mutations may be for a signature for a tumor or cancer in the test subject, i.e., they may be present in cancer cells or pre-cancerous cells of the test subject but not in normal cells of the test subject.

### Amplification of Nucleic Acids

Methods of the invention include amplifying nucleic acids that contain fusion passenger mutations. In some embodiments, one or more nucleic acids containing fusion passenger mutations are amplified by PCR using primers that bind to sequences flanking the fusion site, as described above. Any suitable type of PCR may be used. For example and without limitation, the PCR may be asymmetric PCR, hot-start PCR, ligation-mediated PCR, methylation-specific PCR (MSP), multiplex PCR, nested PCR, quantitative PCR, quantitative real-time PCR (QRT-PCR), reverse transcription PCR (RT-PCR), suicide PCR, or touchdown PCR. PCR methods are known in the art and described in, for example, Green, M R and Sambrook, J, eds., Molecular Cloning, A Laboratory Manual, Fourth Edition, Cold Spring Harbor Laboratory Press 2012, ISBN-13: 978-1936113415; Van Pelt-Verkuil, E., et al., Principles and Technical Aspects of PCR Amplification, Springer; 2008, ISBN-13: 978-1402062407; and Carl W. Dieffenbach and Gabriela S. Dveksler, eds., PCR Primer: A Laboratory Manual, Cold Spring Harbor Laboratory Press 2003, ISBN-13: 978-0879696542, the contents of each of which are incorporated herein by reference.

The nucleic acid may be DNA or RNA. The nucleic acid may by a subpopulation of DNA or RNA. For example and without limitation, the DNA may be cell-free DNA (cfDNA), circulating tumor DNA (ctDNA), or circulating cell-free mitochondrial DNA (ccf mtDNA). For example and without limitation, the RNA may mRNA, tRNA, rRNA, or snRNA.

The one or more amplified nucleic acids may have been previously detected in the subject, or they may not have been previously detected in the subject. The one or more amplified nucleic acids may be detectable in a sample obtained from the subject prior to treatment of the subject for cancer, or they may not be detectable in a sample obtained from the subject prior to treatment of the subject for cancer. The one or more amplified nucleic acids may be detectable in a sample obtained from the subject following treatment of the subject for cancer, or they may not be detectable in a sample obtained from the subject following treatment of the subject for cancer.

The methods may include amplification of multiple nucleic acids in a single reaction or assay. For example, the method may include amplifying 2, 3, 4, 5, 6, 7, 8, 9, 10, 11, 12, 13, 14, 15, 16, 17, 18, 19, 20, about 25, about 30, about 40, about 50, or more nucleic acids in a single reaction or assay. Each nucleic acid may contain a passenger mutation containing or resulting from a fusion. Each nucleic acid may contain a different passenger mutation. Alternatively or additionally, two or more of the nucleic acids may contain the same passenger mutation, i.e., two or more of the nucleic acids may overlap.

### Detection of Nucleic Acids

The methods may include detecting the nucleic acid. For example and without limitation, detection may include chromatography, DNA staining, electron microscopy, electrophoresis, fluorescence (e.g., fluorescence imaging, fluorescence microscopy, fluorescent probe hybridization, or fluorescence resonance energy transfer), immunomagnetic separation, optical microscopy, sequencing, spectrophotometry, or combinations thereof. Methods of detecting nucleic acids are known in the art and described in, for example, Green, M R and Sambrook, J, eds., Molecular Cloning, A Laboratory Manual, Fourth Edition, Cold Spring Harbor Laboratory Press 2012, ISBN-13: 978-1936113415; Van Pelt-Verkuil, E., et al., Principles and Technical Aspects of PCR Amplification, Springer; 2008, ISBN-13: 978-1402062407; and Carl W. Dieffenbach and Gabriela S. Dveksler, eds., PCR Primer: A Laboratory Manual, Cold Spring Harbor Laboratory Press 2003, ISBN-13: 978-0879696542; Peterson, 2009, Generations of sequencing technologies, Genomics 93(2):105-11; Goodwin, 2016, Coming of age: ten years of next-generation sequencing technologies, Nat Rev Genet 17(6):333-51; Morey, 2013, A glimpse into past, present, and future DNA sequencing, Mol Genet Metab 110(1-2):3-24; Xu, 2014, Label-Free DNA Sequence Detection through FRET from a Fluorescent Polymer with Pyrene Excimer to SG, ACS Macro Lett 3(9):845-848; Safarik and Safarikova, Magnetic techniques for the isolation and purification of proteins and peptides, Biomagn Res Technol. 2004; 2:7, doi: 10.1186/1477-044X-2-7; Ballou, David P.; Benore, Marilee; Ninfa, Alexander J. (2008) Fundamental laboratory approaches for biochemistry and biotechnology (2nd ed.), Hoboken, N.J.: Wiley, p. 129. ISBN 9780470087664; Striegel, A. M. et al., Modern Size Exclusion Chromatography, Practice of Gel Permeation and Gel Filtration Chromatography, 2nd ed., Wiley: NY, 2009; Small, Hamish (1989), Ion chromatography, New York: Plenum Press, ISBN 0-306-43290-0; Tatjana Weiss, and Joachim Weiss (2005), Handbook of Ion Chromatography, Weinheim: Wiley-VCH, ISBN 3-527-28701-9; Gjerde, Douglas T. and Fritz, James S. (2000), Ion Chromatography, Weinheim: Wiley-VCH, ISBN 3-527-29914-9; Jackson and Haddad (1990), Ion chromatography: principles and applications, Amsterdam: Elsevier, ISBN 0-444-88232-4, Cady, 2003, Nucleic acid purification using microfabricated silicon structures, Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; Wolfe, 2002, Toward a microchip-based solid-phase extraction method for isolation of nucleic acids, Electrophoresis 23:727-733; and U.S. Pat. No. 8,318,445, the contents of each of which are incorporated herein by reference.

### Samples

In methods of the invention, a nucleic acid is amplified from a sample obtained from the subject. Any sample that contains a nucleic acid may be used. For example and without limitation, the sample may be or include one or more of bile, blood, bone marrow, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, and vomit. The sample may be a tissue sample from an animal. The tissue sample may be from the skin, conjunctiva, gastrointestinal tract, respiratory tract, vagina, placenta, uterus, oral cavity or nasal cavity.

The sample may be obtained by any method. For example and without limitation, the sample may be obtained by aspiration with a needle, liquid biopsy, or tissue biopsy.

In embodiments of the invention, two or more samples of the same type are obtained from the subject at different time points. For example, a test sample may be compared with one or more reference samples obtained from the same subject at earlier time points. The reference samples may have been obtained from the subject before initiation of the prior treatment, during the prior treatment, or subsequent to the prior treatment but prior to obtaining the test sample.

In embodiments of the invention, two or more samples of the same type of are obtained from different subjects. For example, a test sample may be obtained from a subject who has undergone treatment for a disease, and one or more reference samples may be obtained from other subjects. The other subjects may subjects who have not been diagnosed with the disease, subjects who have been diagnosed with the disease but not treated for it, or subjects who have been diagnosed with the disease and treated for it.

In embodiments of the invention in which multiple samples are used, the method may include multiple amplification steps. Thus, the methods may include obtaining multiple samples and independently amplifying nucleic acids from the samples independently. The samples may be processed in parallel. For example, different samples from different subjects may be processed at the same time. Alternatively or additionally, different samples from different subjects may be processed sequentially. For example, a series of samples may be obtained from a test subject at different times, and each sample may be processed after it is obtained and before the next sample is obtained.

### Diseases

Methods of the invention are particularly useful for detecting residual disease in subjects that have already received treatment for the disease. The utility of the methods does not depend on the nature of the prior treatment. Thus, the prior treatment may be treatment by any method for any duration. For example, in embodiments in which the disease is cancer, the prior treatment may be or include one or more of angiogenesis inhibitor therapy, chemotherapy, hormonal therapy, immunotherapy, radiation therapy, surgery, or a targeted therapy, e.g., monoclonal antibody therapy, peptide therapy, photodynamic therapy, or ultrasound therapy. The prior treatment may have been completed, or it may be ongoing.

The subject, e.g., a test subject or reference subject, may or may not have clinical signs of the disease following the prior treatment. For example, the disease may be active, in partial remission, or in complete remission. The disease may have been in remission, e.g., partial remission or complete remission, in the subject for a defined period prior to obtaining the sample for analysis. For example, the disease may have been in remission in the subject for about 3 months, about 6 months, about 9 months, about 1 year, about 2 years, about 3 years, about 4 years, about 5 years, about 6 years, about 8 years, about 10 years, about 15 years, about 20 years, or more.

The methods of the invention are useful for detecting residual cancer. The cancer may be any type of cancer associated with passenger mutations that contain or result from a fusion. For example and without limitation, the cancer may be breast cancer, colon cancer, gastric cancer, glioblastoma, leukemia, liposarcoma, liver cancer, lung cancer, lymphoma, medullablastoma, melanoma, oligoastrocytoma, oligodendroglioma, ovarian cancer, pancreatic cancer, prostate cancer, sarcoma, or thyroid cancer.

### Reports

In certain embodiments, the methods include providing a report containing information based on analysis of the amplified passenger mutations. The report may contain information on one or more of the following: identify of fusion passenger mutations in the sample; number of fusion passenger mutations in the sample; estimate of the subject's tumor mutational burden; efficacy of the subject's prior treatment; development of resistance to a therapy or therapeutic agent in the subject's prior treatment; prognosis for course of disease in the subject; probability that the disease or a particular clinical manifestation of the disease will return for the subject; likelihood that subject will develop resistance to a subsequent therapy or therapeutic agent; and a suggested course of the therapy for the subject. The suggested course of the therapy may have one or more elements that are different from the subject's prior treatment. For example and without limitation, the element of a course of therapy may be or include type of therapy, therapeutic agent, drug dosage, frequency of administration, duration.

Enrichment of Nucleic Acids that Contain or Result from Fusion Passenger Mutations

Methods of the invention may include enriching sample for nucleic acids that contain fusion passenger mutations. The enrichment may be performed prior to amplification to facilitate detection of very rare cancer cells, which may not otherwise be detectable. Alternatively or additionally, the enrichment may be performed subsequent to amplification as a secondary analytical step to confirm results from the amplification analysis and/or to obtained further information about the fusion passenger mutations present in a sample.

Nucleic acids containing fusion passenger mutations may be enriched by using programmable nuclease, such as a CRISPR-associated (Cas) endonuclease, zinc-finger nuclease (ZFN), transcription activator-like effector nuclease (TALEN), or RNA-guided engineered nuclease (RGEN). Programmable nucleases can be engineered to bind to specific DNA sequences. Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference.

One approach involve the use of a programmable nuclease to enrich a sample for fusion passenger mutations is to target a programmable nucleases to sequences on each side of a fusion site and then use an exonuclease to digest DNA that is not bound by the programmable nuclease. Most DNA sequences are degraded by the exonuclease, but the fusion site, which is flanked by the bound programmable nuclease, is protected from degradation. Another approach is to target a programmable nuclease to bind a sequence that comprises that fusion site and then use an exonuclease to digest DNA that is not bound by the programmable nuclease. Here again, the fusion site is protected from degradation by binding of the programmable nuclease. These approaches are described in detail in, for example, International Patent Publication Nos. WO 2018/231945; WO 2018/231946; and WO 2018/231963, the contents of each of which are incorporated herein by reference.

Programmable nuclease generally are able to cleave DNA at or near the sites to which they bind. Cleavage of the target nucleic acid may inhibit detection. Therefore, in certain embodiments, the programmable is enzymatically inactive. The use of enzymatically inactive programmable nucleases is described in, for example, International Patent Publication Nos. WO 2018/231945; WO 2018/231946; and WO 2018/231963, the contents of each of which are incorporated herein by reference.

Any suitable exonuclease may be used to digest unprotected nucleic acids. For example, the exonuclease may be Lambda exonuclease, RecJf, Exonuclease III, Exonuclease I, Exonuclease T, Exonuclease V, Exonuclease VII, T5 Exonuclease, or T7 Exonuclease. Combinations of exonucleases may be used.

Another approach to enrich for fusion passenger mutations is to amplify nucleic acids using modified nucleotides and then use an exonuclease to digest unmodified nucleic acids. Modified nucleic acids, such as DNA having nucleotides joined by phosphorothioate linkages, are not substrates for exonucleases and thus are protected from degradation. One or more of the aforementioned exonucleases may be used to digest unmodified nucleic acids. The use of modified nucleotides to enrich for nucleic acids is described in, for example, International Patent Publication Nos. WO 2018/231955; WO 2018/231967; and WO 2018/231985, the contents of each of which are incorporated herein by reference.

Nucleic acids enriched by the foregoing processes may be further enriched by purification. For example, nucleic acids may be purified by chromatography, electrophoresis, immunomagnetic purification.

Following enrichment of the nucleic acid that contains or results from a fusion passenger mutation, the nucleic acid may be detected. Any of the detection methods described above may be used.

### Kits

The invention also provides kits for performing the methods of the invention. The kit may include any reagent or material useful for performing the methods. For example, the kit may include primers that bind to sequences that flank fusion passenger mutations. The kit may include reagents for enrichment steps, such as guide RNAs for use with Cas endonucleases and nucleic acids, such as DNA or mRNA, that encode a Cas endonuclease. The kit may include materials for sample preparation. The kit may include instructional materials for performing the methods.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method of detecting residual cancer in a subject, the method comprising:
obtaining a sample from a subject that has been treated for cancer; and
amplifying from the sample at least one nucleic acid that comprises a passenger mutation comprising a fusion, thereby detecting residual cancer in the subject.

2. The method of claim 1, wherein the passenger mutation is selected from group consisting of a deletion, inversion, and translocation.

3. The method of claim 1, wherein the amplifying step comprises a first primer that is complementary to a first sequence in the at least one nucleic acid and a second primer that is complementary to a second sequence in the at least one nucleic acid, the first sequence and second sequence flanking the fusion.

4. The method of claim 3, wherein the amplifying step comprises a polymerase chain reaction.

5. The method of claim 1, wherein the nucleic acid is DNA.

6. The method of claim 5, wherein the DNA is cell-free DNA.

7. The method of claim 1, wherein the sample is selected from the group consisting of bile, blood, bone marrow, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, and vomit.

8. The method of claim 1, wherein the obtaining step comprises a liquid biopsy.

9. The method of claim 1, wherein the amplifying step comprises amplifying a plurality of nucleic acids, each of the plurality of nucleic acids comprising a passenger mutation comprising a fusion.

10. The method of claim 1, wherein the cancer is selected from the group consisting of breast cancer, colon cancer, gastric cancer, glioblastoma, leukemia, liposarcoma, liver cancer, lung cancer, lymphoma, medullablastoma, melanoma, oligoastrocytoma, oligodendroglioma, ovarian cancer, pancreatic cancer, prostate cancer, sarcoma, and thyroid cancer.

11. The method of claim 1, further comprising detecting the at least one nucleic acid.

12. The method of claim 11, wherein the detecting step comprises one selected from the group consisting of electrophoresis, chromatography, and fluorescence.

13. The method of claim 1, further comprising enriching the sample for the at least one nucleic acid.

14. The method of claim 13, wherein the enriching step comprises a Cas endonuclease and a guide RNA.

15. The method of claim 1, further comprising providing a report comprising information about the residual cancer in the subject.

16. The method of claim 15, wherein the information comprises one selected from the group consisting of therapeutic efficacy of a prior cancer treatment in the subject, a suggested course of therapy for the subject, and a prognosis for the subject.

17. The method of claim 1, wherein the at least one nucleic acid is detectable in a sample obtained from the subject prior to treatment of the subject for cancer.

18. The method of claim 1, wherein the at least one nucleic acid is not detectable in a sample obtained from the subject prior to treatment of the subject for cancer.

19. The method of claim 1, wherein the at least one nucleic acid is not detectable in a sample obtained from the subject following treatment of the subject for cancer.

20. The method of claim 1, wherein the passenger mutation is associated with cancer in another subject.
